(19) United States
(12) Patent Application Publication
(10) Pub. No.: US 2022/0288206 A1
(43) Pub. Date: Sep. 15, 2022

(54) NANOPARTICLES FOR THE TREATMENT OF CANCER BY RADIOFREQUENCY RADIATION

(71) Applicants: NATIONAL RESEARCH NUCLEAR UNIVERSITY MEPHI, Moscow (RU); NH THERAGUIX, Meylan (FR)

(72) Inventors: Victor TIMOSHENKO, Moscow (RU); Alexander KHARIN, Povarovo (RU); Volodymyn LYSENKO, Villeurbanne (FR); Simon CHAMPAGNE, Lyon (FR); François LUX, Lyon (FR); Paul ROCCHI, Lyon (FR); Olivier TILLEMENT, Fontaines Saint Martin (FR)

(21) Appl. No.: 17/635,116

(22) PCT Filed: Aug. 13, 2020
(86) PCT No.: PCT/FR2020/051466

§ 371 Date: Feb. 14, 2022

(30) Foreign Application Priority Data
Aug. 14, 2019 (FR) 19 09203

(51) Int. Cl. A61K 41/00; A61K 47/69; A61K 33/24; A61K 47/54; A61P 35/00

(52) U.S. Cl. CPC A61K 41/0038; A61K 47/6935; A61K 33/24; A61K 47/547; A61P 35/00

(57) ABSTRACT

The present disclosure relates to a method for treating tumours. In particular, the invention relates to a new therapeutic use of nanoparticles as a sensitiser agent to radiofrequency radiation. More particularly, the invention relates to the use of nanoparticles in combination with radiofrequency radiations for the treatment of tumours, the radiofrequencies inducing hyperthermia of said tumour comprising the nanoparticles in the patient.

### TECHNICAL FIELD

The present disclosure relates to a method for treating tumours. In particular, the invention relates to a new therapeutic use of nanoparticles as a sensitiser agent to radiofrequency radiation. More particularly, the invention relates to the use of nanoparticles in combination with radiofrequency radiations for the treatment of tumours, the radiofrequencies inducing hyperthermia of said tumour comprising the nanoparticles in the patient.

### PRIOR ART

Despite great advances in the treatment of cancers, the treatments used in particular in solid tumours have many considerable and even deleterious side effects.

The use of radiofrequency is increasingly proposed as an alternative treatment.

Radio waves easily penetrate the different tissues and deep areas can be reached. The waves locally induce ionic agitation which triggers molecular frictional movements responsible for a thermal rise which is transmitted in the adjacent tissues leading to an increase in the internal temperature of the tissues resulting in the damage or even the death of the cells. Hence, radiofrequency can be used either to induce a localised hyperthermia in the tumour via a specific probe leading to the ablation of the tumour cells, or to allow making the tumour more sensitive to some treatments.

Although tumour cells are more sensitive to temperature changes than healthy cells, the radiofrequency radiation treatment does not allow targeting a very specific area.

To overcome this drawback, agents allowing absorbing the energy of the waves and increasing local hyperthermia can be used. These agents, called sensitiser agents are for example silicon or gold nanoparticles or carbon nanotubes (Tamarov K P et al. 2014, Scientific Reports, 4: 7034; Rejinov N. J. et al. 2015, Journal of Controlled Release, 204: 84-97). The agents are inserted into the tumours and thus, following the treatment by radiofrequency radiation, allow for a local increase in the temperature specifically at the tumour cells.

However, these agents have many drawbacks. They are large in size and do not specifically target the tumour cells. Hence, these agents must be injected into the tumour. Moreover, these agents are barely biocompatible and difficult to eliminate. In addition, these agents are not suitable for intravenous administration.

Hence, there is still a need to develop sensitiser agents that do not have all of these drawbacks.

### SUMMARY OF THE INVENTION

Surprisingly, the inventors have shown that nanoparticles comprising a non-conductive and non-magnetic matrix functionalised at the surface by metal cations such as gadolinium can interact favourably with radiofrequencies and cause a local increase in temperature, in particular of the cancer cells comprising these nanoparticles and thus block the tumour growth.

Thus, there is provided a nanoparticle for use in the treatment of a tumour by a radiofrequency radiation in a patient inducing a hyperthermia of said tumour, characterised in that said nanoparticle comprises a non-conductive and non-magnetic matrix and metal cations having an atomic number Z greater than 40, said nanoparticle being administered before said treatment with a radiofrequency radiation. Preferably, said matrix is a polysiloxane matrix. Advantageously, said nanoparticle for use as described before comprises at least one chelating agent, preferably DOTA, DTPA, DOTAGA or one of the derivatives thereof intended to complex the metal cations. In a particular embodiment, the metal cations of said nanoparticle represent more than 10% of the mass of said nanoparticle, preferably less than 50% of the mass of said nanoparticle, even more preferably, the metal cations are disposed at the surface of said matrix. Preferably, said metal cations are gadolinium or bismuth. Advantageously, the nanoparticle for use as described before has a size smaller than 10 nm, preferably smaller than 5 nm.

In particular, the nanoparticle is used for the treatment of a tumour, preferably solid, and advantageously selected from the group consisting of a kidney tumour, a lung tumour, a liver tumour, a breast tumour, a bone tumour, said nanoparticle preferably being in a form suitable for administration intravenously, intratumourally or by inhalation.

The invention also relates to a radiofrequency hyperthermal sensitiser agent comprising said nanoparticle as described before.

### BRIEF DESCRIPTION OF THE DRAWINGS

Other features, details and advantages will appear upon reading the detailed description hereinafter, and upon analysing the appended figures, wherein:

FIG. 1 represents the transition temperature of deionised water (black square), of a saline medium (triangle), of a saline medium in the presence of albumin (inverted triangle) and of the AGuIX solution (lozenge) under treatment with a radiofrequency radiation at 27 MHz.

FIG. 2 represents the transition temperature of deionised water, of a medium comprising yttrium, gadolinium, bismuth, terbium, AGuIX under treatment with a radiofrequency radiation at 27 MHz.

FIG. 3 represents MRI images of mice before and after intratumoural injection of a saline solution of AGuIX.

FIG. 4 represents thermal images of a mouse during treatment with a radiofrequency radiation for 1, 5 and 10 min.

FIG. 5 is a graph (A) representing the size of the tumour at different times after treatment with a radiofrequency radiation of the different groups of control mice (black circle), injected intratumourally with a solution comprising AGuIX (circle), injected intratumourally with a solution comprising AGuIX followed by a radiofrequency treatment (square), (B) representing the size of the tumour after the radiofrequency treatment of mice injected intratumourally with a saline followed by a radiofrequency treatment (square), and mice injected intratumourally with a solution comprising AGuIX followed by a treatment with a radiofrequency radiation (circle).

FIG. 6 is a graph representing survival at different times after Lewis lung carcinoma transplantation of different groups of mice injected intratumourally with a saline solution (black square), injected intratumourally with a saline followed by a radiofrequency treatment (inverted triangle), injected intratumourally with a solution comprising AGuIX (circle), injected intratumourally with a solution comprising AGuIX followed by a radiofrequency treatment (triangle).

### DESCRIPTION OF THE EMBODIMENTS

The inventors have shown that the administration of nanoparticles comprising a non-magnetic and non-conductive polymer matrix and metal cations having an atomic number greater than 40 leads to a decrease in the tumour growth in vivo in mice.

The nanoparticles according to the invention are deposited in the tumour and will act as a sensitiser agent to the radiofrequency treatment. Indeed, following the radiofrequency treatment, the nanoparticles present in the tumour will absorb a large amount of energy and cause a greater energy dissipation leading to a local hyperthermia in the tumour and in the elimination of the tumour cells.

By hyperthermia, it should be understood temperatures higher than the body temperature, in particular above 37° C. in humans.

In a particular embodiment, by hyperthermia, it should be understood a local body temperature comprised between 37.5° C. and 45° C., preferably 39 and 45° C. Hyperthermia will allow eliminating or damaging the target cells or sensitising them for another treatment, especially radiotherapy or chemotherapy.

### Nanoparticles

Thus, the present invention relates to nanoparticles comprising a non-magnetic and non-conductive matrix and metal cations having an atomic number Z greater than 40 for use in the treatment of a tumour by a radiofrequency radiation in a patient, said nanoparticle being administered before said treatment with a radiofrequency radiation.

Nanoparticles are particles with a size in the range of nanometres.

In a particular embodiment, the nanoparticles are administered to the subject via the intravenous route. In this case, the nanoparticles must be small enough to be able to target the tumour cells via the vascular system and be quickly eliminated by the kidneys. Thus, in a particular embodiment of the invention, the nanoparticles have a diameter smaller than 20 nm, preferably smaller than 10 nm.

More particularly, the nanoparticles are particles whose average diameter is comprised between 1 and 20 nm, preferably between 1 and 10 nm and even more preferably between 2 and 5 nm, or even between 1 and 6 nm.

According to the invention, nanoparticles with a very small diameter, for example comprised between 1 and 10 nm, preferably between 2 and 5 nm, will advantageously be used.

For example, the size distribution of the nanoparticles is measured using a commercial particle size analyser, such as a Malvern Zetasizer Nano-S particle size analyser based on PCS (Photon Correlation spectroscopy). This distribution is characterised by an average hydrodynamic diameter.

In the context of the invention, by “average diameter”, it should be understood the harmonic mean of the diameters of the particles. A method for measuring this parameter is also described in the standard ISO 13321:1996.

The nanoparticles according to the invention are nanoparticles comprising an organic or hybrid (organic-inorganic) non-magnetic and non-conductive matrix.

By non-conductive matrix, it should be understood an insulating matrix, i.e. a matrix that does not conduct electricity. Preferably, the matrix does not contain conductive materials such as metals in their metallic form (in the zero oxidation state).

By non-magnetic matrix, it should be understood a matrix that is not attracted to the magnetic field. Advantageously, the nanoparticle according to the invention comprises a non-ferromagnetic and/or non-super-paramagnetic matrix, and preferably comprises no or less than 5% of iron, cobalt or nickel by mass of the matrix.

Preferably, the nanoparticle comprises a non-magnetic and non-conductive matrix that is a biocompatible polymer such as polyethylene glycol, polyethylene oxide, polyacrylamide, biopolymers, polysaccharides or polysiloxane, preferably polysiloxane.

The nanoparticles as described before further comprise metal cations having an atomic number greater than 40, allowing acting as sensitiser agents at radiofrequencies. More particularly, the metal cations are chosen from heavy metals, preferably from the group consisting of: Pt, Pd, Sn, Ta, Zr, Tb, Tm, Ce, Dy, Er, Eu, La, Nd, Pr, Lu , Yb, Bi, Hf, Ho, Sm, In and Gd, or a mixture thereof. Preferably, the metal cations are Bi and/or Gd.

Preferably, the nanoparticle for use according to the invention has a metal cation mass ratio, in particular of Bi and/or Gd, of more than 10%, preferably comprised between 10 and 50%.

The metal cations could be coupled to the matrix by covalent couplings or trapped by a non-covalent bond, for example by encapsulation or hydrophilic/hydrophobic interaction or using a chelating agent.

In a preferred embodiment, the metal cations are located at the surface of the matrix of the nanoparticle.

Preferably, the nanoparticles that could be used according to the invention comprise chelating agents which are covalently bonded to the matrix and allow complexing the metal cations. Preferably, the chelating agents are grafted at the surface of the matrix of the nanoparticle so as to complex the metal cations at the surface of the matrix.

Preferably, the nanoparticle for use according to the invention comprises a polysiloxane matrix, a chelating agent covalently bonded to said matrix and a metal cation complexed by the chelating agent.

Advantageously, the chelating agent is chosen from the following products:
products from the group of polyamino polycarboxylic acids and derivatives thereof and more preferably from the subgroup comprising: DOTA (1,4,7,10-tetraazacyclododecane-N,N′,N″,N″′-teracetic acid), DTPA (diethylene triamine penta-acetic acid), EDTA (2,2′,2″,2′″-(ethane-1,2-diyldinitrilo)tetraacetic acid), EGTA (ethylene glycol-bis(2-aminoethyl ether)-N,N,N′,N′-tetraacetic acid), BAPTA (1,2-bis(o-aminophenoxy)ethane-N,N,N′,N′-tetraacetic acid), NOTA (1,4,7-triazacyclononane-1,4,7-triacetic acid), DOTAGA (2-(4,7,10-tris(carboxymethyl)-1,4,7,10-tetraazacyclododecan-1-yl)pentanedioic acid), DTPABA 2-(bis(2-(2,6-dioxomorpholino)ethyl)amino)acetic acid, the amide derivatives thereof such as, for example, DOTAM (1,4,7,10-tetrakis(carbamoylmethyl)-1,4,7,10 tetraazacyclododecane) or NOTAM (1,4,7-tetrakis (carbamoylmethyl)-1,4,7-triazacyclononane), the phosphonic derivatives thereof such as DOTP (1,4,7,10-tetraazacyclododecane1,4,7,10-tetrakis(methylene phosphonate)) or NOTP (1,4,7-tetrakis (methylene phosphonate)-1,4,7-triazacyclononane) and mixtures thereof,
the products from the group comprising porphyrin, chlorine, 1,10-phenanthroline, bipyridine, terpyridine, cyclam, triazacyclononane, and derivatives thereof, such as derivatives of cyclam such as TETA (1,4,8,11-tetraazacydotetradec ane-N,N′,N′,N″′-tetraacetic acid), TETAM (1,4,8,11-tetraazacyclotetradec ane-N,N′,N″,N″′-tetrakis (carbamoylmethyl)), TETP (1,4,8,11-tetraazacyclotetradecane-N,N′,N″,N′″-tetrakis (methylene phosphonate)), and mixtures thereof,
and mixtures thereof.

According to a particular embodiment, the chelating agent is chosen from DOTA, DTPA, EDTA, EGTA, BAPTA, NOTA, DOTAGA, DTPABA, DOTAM, DOTP, NOTP and mixtures thereof.

In a preferred embodiment, when the nanoparticle comprises a metal cation Gd or Bi, the chelating agent is DOTA, DTPA, DOTAGA or one of the derivatives thereof, preferably DTPA, DOTAGA or one of the derivatives thereof such as for example DOTAM or NOTAM.

In a particular and preferred embodiment, the ratio of metal cations per nanoparticle, for example the ratio of rare-earth elements, for example gadolinium (optionally chelated with DOTAGA) per nanoparticle is between 3 and 100, preferably between 5 to 20 , typically around 10.

If the metal cation is a lanthanide, for example gadolinium, the chelating agent is advantageously selected from those whose log(KC1) complexation constant is greater than 15, preferably 20. As preferred examples of chelating agents, complexing lanthanides, mention may be made of those comprising a diethylene triamine penta acetic acid (DTPA) unit, 1,4,7,10-tetraazacyclododec ane-1,4,7,10-tetra acetic acid (DOTA) or 1,4,7,10-tetraazacyclododecance-1,glutaric anhydrous-4,7,10-triacetic acid (DOTAGA).

### “Coreless” functionalised ultrafine nanoparticles

In a more particularly preferred embodiment, in particular because of their very small size, the nanoparticles that could be used according to the invention are obtained by the following method:
obtain a core comprising a metal oxide, M being a metal element,
add at least one coating layer (shell) comprising polysiloxanes, for example by a sol gel method;
graft a chelating agent to the layer of polysiloxanes, the chelating agent being bound to said polysiloxane layer by a —Si—C— covalent bond, to obtain a core-shell precursor nanoparticle
purify and transfer the core-shell precursor nanoparticle into an aqueous solution in which the grafting agent is in a sufficient amount to dissolve the metal oxide core and to complex the metal cation so that the average diameter of the nanoparticle thus obtained is reduced to a value of less than 10 nm, preferably less than 5 nm, for example between 1 and 5 nm.

These nanoparticles obtained according to the embodiment described hereinabove do not comprise a core embedded by at least one coating. Further details on the synthesis of these nanoparticles are given in the next section.

This results in nanoparticles with observed sizes comprised between 1 and 8 nm, for example between 1 and 5 nm. We then talk about ultrafine nanoparticles.

Alternatively, another “one-pot” synthesis method enabling the preparation of coreless nanoparticles with an average diameter of less than 10 nm, typically between 1 and 8 nm, for example between 1 and 5 nm, is described in the next section.

The chelating agents could be grafted onto the surface of the polysiloxane particles or directly inserted into the POS matrix. Some or all of these chelating agents are intended to complex metal cations (e.g. gadolinium, bismuth).

Besides the chelating functionalisation, these nanoparticles could be modified (functionalisation) at the surface by hydrophilic compounds (PEG) and/or charged differently to adapt their bio-distribution within the organism and/or to allow for a good cellular marking, in particular for monitoring cell therapies.

For example, they could be functionalised at the surface by grafting molecules targeting the lung tissues, or, because of their passage in the blood, by grafting molecules targeting some areas of interest of the organism, in particular tumour areas.

The functionalisation could also be done by compounds including another active principle and/or luminescent compounds (fluorescein). This results in possibilities of therapeutic uses as a radiosensitiser agent, neutron therapies, as a radioactive agent for brachytherapy treatments, as an agent for PDT (photodynamic therapy) or as an agent for vectorising molecules with a therapeutic effect.

Another characteristic of these ultrafine nanoparticles is the maintenance of the rigid nature of the objects and of the overall geometry of the particles after injection. This strong three-dimensional rigidity could be ensured by a polysiloxane matrix, where most of the silicons are bonded to 3 or 4 other silicon atoms via an oxygen bridge. The combination of this rigidity with their small size allows increasing the relaxivity of these nanoparticles for intermediate frequencies (20 to 60 MHz) in comparison with commercial compounds (complexes based on Gd-DOTA for example), but also for frequencies higher than 100 MHz present in new-generation high-field MRIs.

This rigidity, not present in polymers, is also an advantage for the vectorisation and accessibility of the targeting molecules.

Preferably, the nanoparticles according to the invention, and in particular according to the present embodiment, have a relaxivity r₁ per Mⁿ⁺ metal cation which is greater than 5 mM⁻¹·s⁻¹ (of Mⁿ⁺ ion), preferably 10 mM⁻¹·s⁻¹ (of Mⁿ⁺ ion), for a frequency of 20 MHz. For example, they have a relaxivity r₁ per nanoparticle comprised between 50 and 5000 mM⁻¹·s⁻¹. Still better, these nanoparticles have a relaxivity r₁ per Mⁿ⁺ ion at 60 MHz which is greater than or equal to the relaxivity r₁ per Mⁿ⁺ ion at 20 MHz. The relaxivity r₁ considered herein is a relaxivity per Mn ion (for example gadolinium). r₁ is extracted from the following formula: 1/T₁=[1/T₁]_{water}+r₁[Mⁿ⁺].

More details regarding these ultrafine nanoparticles, their methods of synthesis and their applications are described in the patent application WO2011/135101, WO2018/224684 or WO2019/008040 which are incorporated herein for reference.

According to a preferred embodiment, the nanoparticles that could be used according to the invention are polysiloxane nanoparticles chelated with gadolinium. In particular, they consist of polysiloxane nanoparticles chelated with gadolinium, which do not comprise a gadolinium oxide core and whose diameter is comprised between 1 and 10 nm, preferably between 2 and 8 nm. In particular, such nanoparticles are the so-called AGuIX nanoparticles of general formula I hereinbelow:

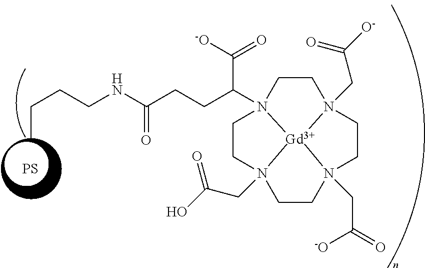

wherein PS is a polysiloxane matrix and n is comprised between 5 and 50, preferably between 5 and 20, and wherein the hydrodynamic diameter is comprised between 1 and 10 nm, for example between 2 and 8 nm, in particular 5 nm.

According to this embodiment, the AGuIX nanoparticles may have a mass of about 15 kDa±10 kDa.

Still according to this preferred embodiment, the AGuIX nanoparticles may also be described by formula II hereinafter:

(GdSi₃₋₈C₂₄₋₃₄N₅₋₈O₁₅₋₃₀H₄₀₋₆₀, 1-10 H₂O)n

### Nanoparticle Preparation Method

In general, a person skilled in the art can easily manufacture nanoparticles used according to the invention.

As regards the POS matrix, several techniques may be used, derived from those initiated by Stoeber (Stoeber, W; J. Colloid Interf Sci 1968, 26, 62). It is also possible to use the method used for coating as described in Louis et al. (Louis et al., 2005, Chemistry of Materials, 17, 1673-1682) or the international application WO 2005/088314.

In practice, the synthesis of ultrafine nanoparticles is for example described in Mignot et al. Chem. Eur. J. 2013, 19:6122-6136. Typically, a core/shell type nanoparticle is formed with a lanthanide oxide core (through a modified polyol route) and a polysiloxane shell (by sol/gel), this object has for example a size around 10 nm (preferably 5 nanometres). A lanthanide oxide core with a very small size (adaptable to less than 10 nm) could thus be made in an alcohol by one of the methods described in the following publications: P. Perriat et al., J. Coll. Int. Sci, 2004, 273, 191; 0. Tillement et al., J. Am. Chem. Soc, 2007, 129, 5076 and P. Perriat et al., J. Phys. Chem. C, 2009, 113, 4038. These cores could be coated with a layer of polysiloxane by following, for example, a protocol described in the following publications: C. Louis et al., Chem. Mat., 2005, 17, 1673 and O. Tillement et al., J. Am. Chem. Soc, 2007, 129, 5076.

Chelating agents that are specific to the targeted metal cations (for example DOTAGA for Gd³⁺) are grafted at the surface of the polysiloxane; it is also possible to insert a portion thereof inside the layer, but the control of the formation of the polysiloxane is complex and the mere external grafting gives, at these very small sizes, a sufficient grafting proportion.

The nanoparticles are separated from the synthetic residues by a method of dialysis or tangential filtration, on a membrane comprising pores with a suitable size.

The core is destroyed by dissolution (for example by modifying the pH or by bringing in complexing molecules into the solution). This destruction of the core then allows for a scattering of the polysiloxane layer (according to a collapse or slow corrosion mechanism), which ultimately allows obtaining a polysiloxane object with a complex morphology whose characteristic dimensions are in the same order of magnitude as the thickness of the polysiloxane layer, i.e. much smaller than the objects developed so far. Thus, removing the core allows switching from a particle size of about 5 nanometres in diameter into a size of about 3 nanometres. In addition, this operation allows increasing the number of metal cations (e.g. gadolinium) per nm in comparison with a theoretical polysiloxane nanoparticle with the same size but comprising metal (e.g. gadolinium) only at the surface. The number of metal cations for a nanoparticle size could be assessed using the M/Si atomic ratio measured by EDX.

On these nanoparticles, it is possible to graft targeting molecules, for example using coupling by peptide bond on an organic constituent of the nanoparticle, as described in Montalbetti, C.A.G.N, F algue B. Tetrahedron 2005, 61, 10827-10852. It is also possible to use a coupling method using “click chemistry” Jewett, J. C.; Bertozzi, C. R. Chem. Soc. Rev. 2010, 39, 1272-1279, and involving groups of the type:

—N3, —CN, —C≡CH, or one of the following groups:

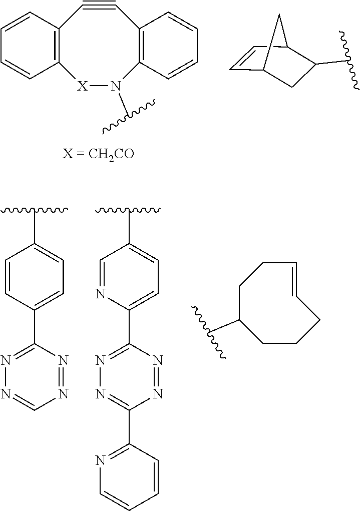

In a specific embodiment, the nanoparticle according to the invention comprises a chelating agent having an acid function, for example DOTA. It is proceeded with the activation of the acid function of the nanoparticle, for example using EDC/NHS (1 -ethyl-3-(3-dimethylaminopropyl)carbodiimide/N-hydrosuccinimide) in the presence of a suitable amount of targeting molecules. The nanoparticles thus grafted are then purified, for example by tangential filtration.

In a particular embodiment, the nanoparticles according to the present invention are obtained by a synthesis method (“one-pot synthesis method”) comprising mixing at least one hydroxysilane or alkoxysilane that is negatively-charged at a physiological pH and of at the least one chelating agent chosen from polyaminopolycarboxylic acids with:
at least one hydroxysilane or alkoxysilane that is neutral at a physiological pH, and/or
at least one hydroxysilane or alkoxysilane that is positively-charged at a physiological pH and comprises an amine function,

wherein:
the molar ratio A of neutral silanes to negatively-charged silanes is defined as follows: 0≤A≤6, preferably 0.5≤A≤2;
the molar ratio B of positively-charged silanes to negatively-charged silanes is defined as follows: 0≤B≤5, preferably 0.25≤B≤3;
the molar ratio C of the positively-charged and neutral silanes to the negatively-charged silanes is defined as follows: 0≤C≤8, preferably 1≤C≤4.

In a more particular embodiment, the “one pot” synthesis method comprises mixing of at least one alkoxysilane that is negatively-charged at a physiological pH, said alkoxysilane being chosen from APTES-DOTAGA, TANED, CEST, and mixtures thereof with:
at least one alkoxysilane that is neutral at a physiological pH, said alkoxysilane being chosen from TMOS, TEOS and mixtures thereof, and/or
APTES that is positively-charged at a physiological pH, wherein:
the molar ratio A of neutral silanes to negatively-charged silanes is defined as follows: 0≤A≤6, preferably 0.5≤A≤2;
the molar ratio B of positively-charged silanes to negatively-charged silanes is defined as follows: 0≤B≤5, preferably 0.25≤B≤3;
the molar ratio C of the positively-charged and neutral silanes to the negatively-charged silanes is defined as follows: 0≤C≤8, preferably 1≤C≤4.

According to a particular embodiment, the “one pot” synthesis method comprises mixing of APTES-DOTAGA that is negatively-charged at a physiological pH with:
at least one alkoxysilane that is neutral at a physiological pH, said alkoxysilane being chosen from TMOS, TEOS and mixtures thereof, and/or
APTES that is positively-charged at a physiological pH, wherein:
the molar ratio A of neutral silanes to negatively-charged silanes is defined as follows: 0≤A≤6, preferably 0.5≤A≤2;
the molar ratio B of positively-charged silanes to negatively-charged silanes is defined as follows: 0≤B≤5, preferably 0.25≤B≤3;
the molar ratio C of the positively-charged and neutral silanes to the negatively-charged silanes is defined as follows: 0≤C≤8, preferably 1≤C≤4.

### Therapeutic Method

The nanoparticles as previously described are administered into the tumour or in the proximity of the region of the tumour of a patient. They may also be administered by intravenous, intramuscular injection or by inhalation. The radiofrequency radiation treatment of the patient then induces hyperthermia of said tumour and reduces the tumour growth.

The nanoparticles as defined before are used as an sensitiser agent to radiofrequency radiations to specifically target tumour cells.

The radiofrequency radiation sensitiser agents as used in the present application refer to a composition that allows inducing a greater amount of energy absorption from a radiofrequency signal thereby creating a higher temperature rise in the area comprising this composition. The sensitiser agents are in the present application characterised by their ability to target and bind to a target cell, herein a tumour cell, and allow making the target cell more sensitive to the temperature increase induced by a radiofrequency radiation.

The present invention thus relates to nanoparticles as defined before for use in the treatment of a tumour in a patient undergoing a treatment with a radiofrequency radiation.

By “patient” or “subject”, it should be understood any animal, preferably a mammal or a human being including for example a subject having a tumour.

The terms “treatment”, “therapy”, refer to any act that aims to improve the health condition of a patient, such as therapy, prevention, prophylaxis, and the delay of a disease. In some cases, these terms refer to the improvement or eradication of a disease or the symptoms associated with the disease. In other embodiments, these terms refer to the reduction in the spread or aggravation of the disease resulting from the administration of one or several therapeutic agent(s) to a subject afflicted with such a disease.

In particular, nanoparticles are used for the treatment of solid tumours, in particular brain cancer (primary and secondary, glioblastoma, etc.), liver cancers (primary and secondary), pelvic tumours (cervical cancer, prostate cancer, anorectal cancer, colorectal cancer), upper aerodigestive tract cancers, lung cancer, oesophageal cancer, breast cancer, pancreatic cancer.

The present invention relates to a method for treating tumours with a radiofrequency radiation comprising the steps of administering an effective dose of nanoparticles as described before into the tumour of a patient and exposing the tumour to radiofrequency radiation.

By “effective dose” of nanoparticles, reference is made to the amount of nanoparticles as described before which, when administered to a patient, is sufficient to be localised in the tumour and induce hyperthermia following the treatment with a radiofrequency radiation.

This dose is determined and adjusted according to factors such as the age, sex and weight of the subject.

The administration of the nanoparticles as described before could be carried out by intratumoural, subcutaneous, intramuscular, intravenous, intradermal, intraperitoneal, oral, sublingual, rectal, vaginal, intranasal route, by inhalation or by transdermal application.

The composition is in a galenic form suitable for a chosen administration.

Preferably, the nanoparticles are administered intravenously and the nanoparticles will specifically target the tumours, by passive targeting, for example by increasing the permeability and retention effect.

Repeated administrations could be carried out.

In a particular embodiment, a single dose comprised between 20 mg/kg and 500 mg/kg of nanoparticles is administered intravenously in a subject.

In a particular embodiment, the nanoparticles are administered into the tumour of the patient such that the nanoparticles are present at a concentration comprised between 0.1 mg/L and 50 mg/L, preferably 1 and 10 mg/L in the region of the tumour that will be treated by radiofrequency.

The nanoparticles act as sensitiser agents and are used to specifically target tumour cells. The emission of radio waves in the proximity of tumour cells comprising the nanoparticles then leads to the elimination of the tumour cells.

Methods for treating cancer with a radiofrequency radiation are well known and the parameters used to treat tumours with radiofrequency non-invasively could be optimised by a person skilled in the art.

A radiofrequency radiation induces oscillating motions of the charged species at frequencies in the range of 3 kHz to 300 GHz. Following these electromagnetic excitations, an ionic agitation triggers molecular frictional movements responsible for a thermal rise in the cells. The thermal rise then leads to the elimination of the cells.

A radiofrequency radiation is generated between a transmission head and a reception head different from the transmit head. The transmission and reception heads are disposed on either side of the tumour site or the body of the patient and the radio frequency signal is emitted to induce hyperthermia of the target cells, such as tumour cells. Many devices are known to emit radio waves.

The treatment with a radiofrequency radiation according to the invention is preferably a non-invasive treatment. The term “non-invasive” as used in the present application means that no needle, wire, electrodes or other objects are inserted into the patient or the tumour of the patient which is to be treated.

The radiofrequency signal is emitted such that the target tumour reaches a temperature comprised between 37.5 and 45° C., preferably between 42 and 44° C.

The radiofrequency treatment is carried out at a frequency lower than 1 GHz comprised between 1 and 1000 MHz, preferably between 1 and 100 MHz.

The radiofrequency signal must be high enough to allow inducing the hyperthermia of the tumour cells and thus induce their cell death or at least the damage of the target cells.

The radiofrequency treatment could be carried out through a single exposure or successive exposures to a radiofrequency radiation. In a particular embodiment, the duration of each exposure to a radiofrequency radiation is comprised between 1 and 60 min, preferably between 10 and 60 min.

The frequency and the time of radiofrequency treatment could be optimised, for example, according to the patient, the cancer type, the gender, the size of the individual.

The temperature of the target zone could be measured using a device well known to a person skilled in the art. For example, the temperature could be measured using an infrared camera, a contactless thermometer, a thermal probe or by thermal magnetic resonance imaging. These probes are thermally and electrically inert to the radiofrequency treatment.

In a particular embodiment, the treatment with a radiofrequency radiation may comprise one exposure to a radiofrequency radiation per week, or several exposures per week.

The hyperthermia induced by a radiofrequency radiation will also make the cancer cells more sensitive to radiation therapy or anti-cancer drugs. Thus, the nanoparticles as previously described for use in the treatment of a tumour by a radiofrequency radiation could be used in combination with one or several anti-cancer agent(s) or with radiotherapy.

The chemotherapy agents could consist of DNA replication inhibitors such as DNA binding agents, in particular alkylating or intercalating drugs, antimetabolite agents such as polymerase or topoisomerase I or II inhibitors, or anti-mitotic agents such as alkaloids. Non-limiting examples of chemotherapy agents are: 5-FU, oxaliplatin, cisplatin, carboplatin, irinotecan, cetuximab, erlotinib, docetaxel, doxorubicin and paclitaxel.

Immunotherapy agents are compounds that indirectly or directly improve or stimulate the immune response against the tumour cells.

The nanoparticles could also be further used as a radio-sensitiser agent for radiotherapy, as a photosensitiser agent for phototherapy or as an agent for beam therapy.

Advantageously, the nanoparticles used for the treatment of tumours by radiofrequency are also used as a contrast agent or an imaging agent to visualise the tumour in vivo, by medical imaging enabling for example monitoring of the therapy.

In the context of the invention, the term “contrast agent” refers to any product or composition used in medical imaging in order to artificially increase the contrast allowing visualising a particular anatomical structure (for example some tissues or organs) or a pathological anatomical structure (for example tumours) relative to neighbouring or non-pathological structures. The term “imaging agent” refers to any product or composition used in medical imaging in order to create a signal allowing visualising a particular anatomical structure (for example some tissues or organs) or a pathological anatomical structure (for example tumours) relative to neighbouring or non-pathological structures. The way in which the contrast or imaging agents act depends on the imaging techniques that are used.

Preferably, the medical imaging is chosen from the following techniques: nuclear magnetic resonance, X-ray scanners, fluorescence imaging, SPECT scintigraphy, PET scintigraphy, still more preferably the tumour is visualised in vivo by nuclear magnetic resonance, in particular in dynamic magnetic resonance imaging (MRI) (i.e. DCE standing for Dynamic Contrast Enhanced sequence). In particular, MRI allows obtaining spatio-temporal accuracy that is particularly advantageous for the implementation of the present invention.

An object of the present invention is also a pharmaceutical composition comprising a nanoparticle as defined hereinabove and a pharmaceutically-acceptable vehicle, a carrier substance and/or an adjuvant for use in the treatment of a tumour with a radiofrequency radiation in a patient as previously described. Pharmaceutically-acceptable vehicles, a carrier substance and/or an adjuvant are those conventionally used.

The present disclosure is not limited to the following examples, but it encompasses all variants that a person skilled in the art might consider within the pursued scope.

### EXAMPLES

### 1. Samples

The AGuIX nanoparticles (50 mM per bottle) are obtained by Dr. O. Tillement via Dr. V. Lysenko.

The nanoparticles are dissolved in a physiological solution at a concentration of 20 mM (per Gd).

### 2. Treatment with a Radiofrequency Radiation

The radiofrequency electromagnetic radiations are generated by a medical device UVCH-60 (MedTeeko Ltd., Russia) operating at 27 MHz with a power up to 60 W.

Different cuvettes containing water, a saline solution, a saline solution with 50 g/L albumin and a saline solution with 50 g/L albumin and 7.5 mM of AGuIX nanoparticle (Gd) are treated by a radiofrequency radiation for a period of 20 to 30 min. The temperatures of the solutions are measured without contact using a thermometer.

Afterwards, different cuvettes containing water, Yttrium (Y) (10.3 mM), Gadolinium (Gd) (10.2 mM), Bismuth (Bi) (9.9 mM), Terbium (Tb) (10.5 mM), AGuIX are treated with a radiofrequency radiation as described before.

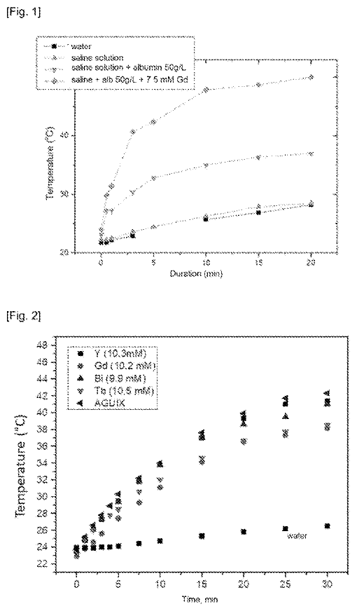

The experiments carried out with 10 mL cuvettes filled with AGuIX nanoparticles and reference liquids show that the treatment with a radiofrequency radiation allows for a greater rise in temperature in the AGuIX solutions than in the reference solutions. The AGuIX nanoparticles act as an important sensitiser agent to treatment with a radiofrequency radiation (FIGS. 1 and 2).

### 3. In Vivo Studies

C57BI/6, BDF1 mice having Lewis lung carcinoma are used. Lewis lung carcinoma transplantation is performed by homogenising Lewis lung carcinoma tumour tissue in a Medium 199 (Merck) sterile solution.

Donor animals are sacrificed, and pieces of tumours are excised without necrotic site and then homogenised in Medium 199. The tumour mass is diluted in Medium 199 and administered intramuscularly into the right hip of C57BI/6 mice in a volume of 0.3 mL.

The mice are divided into four groups, a group of control mice injected with a saline solution (A), a group of mice injected with a saline solution and treated with a radiofrequency radiation for 10 min (B), a group of mice injected with AGuIX and not treated with a radiofrequency radiation (C), and a group of mice injected with AGuIX and treated with a radiofrequency radiation (D) (Table 1).

The saline solutions and AGuIX (0.2 mL) are injected intramuscularly six days after tumour inoculation, when the tumour reaches a size of 70±15 mm³.

All animal experiments are carried out in compliance with the principles of work with laboratory animals (NIH Rules No. 85-23, revised in 1985) and the European Convention for the protection of animals used for experimental purposes or for other scientific purposes (Strasbourg, 18.III.1986, ETS protocol 170).

TABLE 1
description of the different groups of mice

| | Number | |
|---|---|---|
| Group | of mice | Description of the group |
| A | 10 | 0.2 ml of intratumoural saline solution |
| B | 10 | 0.2 ml of intratumoural saline solution followed |
| | | by a radiofrequency (RF) for 10 min |
| C | 10 | 0.2 ml at 20 mM of intratumoural AguIX |
| D | 10 | 0.2 ml at 20 mM of intratumoural AguIX |
| | | followed by a radiofrequency (RF) for 10 min |

### 4. Monitoring by MRI

The monitoring by MRI of the biodistribution of the AGuIX is carried out using a Bruker BioSpec 7 T MRI scanner (Briker BioSpin GmbH, Germany) with a gradient system of 105 mT/m using the ParaVision 5.0 software.

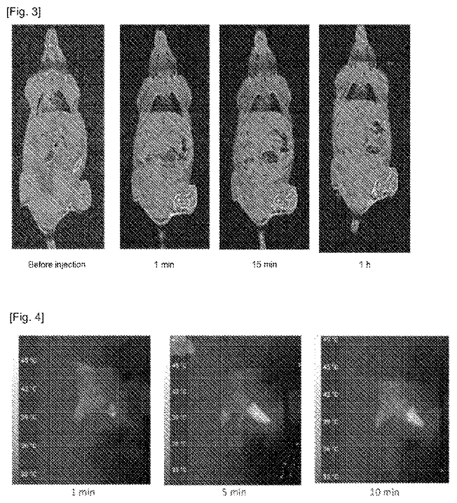

FIG. 3 shows the MRI images of a mouse before and after the intratumoural injection of the AGuIX solution. The AGuIX nanoparticles are observed in the tumour region at least one hour after the injection.

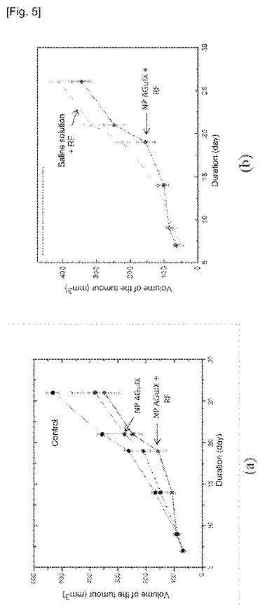

The mice of the groups B and D are treated with a radiofrequency radiation with a power of about 10 W for 10 min. Thermal monitoring of the mice during the treatment with a radiofrequency radiation is carried out with a Seek Thermal thermal camera. A maximum temperature of about 43-45° C. in the tumour is measured 5 to 10 minutes after the start of the radiofrequency treatment (FIG. 4). The survival of the injected mice is then monitored 65 days after the Lewis lung carcinoma transplant. The survival of the mice is improved in the mice injected with AGuIX and treated with a radiofrequency radiation (FIG. 6).

### Conclusions

The AGuIX nanoparticles act as hyperthermia sensitiser agents following the treatment with a radiofrequency radiation. As shown by the MRI data, the AGuIX nanoparticles injected intratumourally are located in the region of the tumour for at least 1 h after the injection.

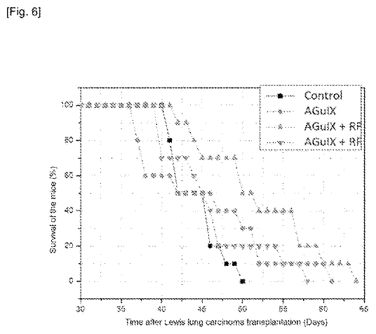

The injection of AGuIX nanoparticles followed by the radiofrequency treatment allows suppressing the growth of Lewis lung carcinoma by hyperthermia (FIG. 5) and improving the survival of the mice (FIG. 6). The growth of Lewis lung carcinoma is monitored using a thermal camera.

To improve the effect of sensitiser agents to hyperthermia by a radiofrequency radiation, different ways of optimisation may be proposed: (i) prolonging the radiofrequency treatment time (more than one hour) after a single injection, (ii) repeated treatments (injection of AGuIX followed by the radiofrequency treatment, (iii) administration of the AGuIX nanoparticles intravenously while monitoring the maximum accumulation in the tumours followed by the treatment with a radiofrequency radiation.

1. A nanoparticle for use in the treatment of a tumour by a radiofrequency radiation in a patient inducing hyperthermia of said tumour, characterised in that said nanoparticle comprises a non-conductive and non-magnetic matrix and metal cations having an atomic number Z greater than 40, said nanoparticle being administered prior to said treatment by a radiofrequency radiation.

2. The nanoparticle for use according to claim 1, characterised in that the nanoparticle comprises a polysiloxane matrix.

3. The nanoparticle for use according to claim 1, characterised in that said nanoparticle comprises at least one chelating agent, preferably DOTA, DTPA, DOTAGA or derivatives thereof, intended to complex the metal cations.

4. The nanoparticle for use according to claim 1, characterised in that the metal cations represent more than 10% of the mass of said nanoparticle and preferably less than 50% of the mass of said nanoparticle.

5. The nanoparticle for use according to claim 1, characterised in that the metal cations are disposed at the surface of said matrix.

6. The nanoparticle for use according to claim 1, characterised in that the metal cations are gadolinium or bismuth.

7. The nanoparticle for use according to claim 1, characterised in that said nanoparticle has a size smaller than 10 nm, preferably smaller than 5 nm.

8. The nanoparticle for use according to claim 1, characterised in that the tumour is selected from the group consisting of kidney tumour, lung tumour, liver tumour, breast tumour, bone tumour.

9. The nanoparticle for use according to claim 1, characterised in that said nanoparticle is in a form suitable for administration intravenously, intratumourally or by inhalation.

10. The nanoparticle for use according to claim 1, characterised in that it has the general formula I hereinbelow: 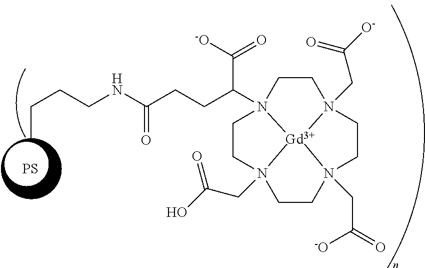
wherein PS is a polysiloxane matrix, and n is comprised between 5 and 50, preferably between 5 and 20,
and wherein the hydrodynamic diameter is comprised between 1 and 10 nm, for example between 2 and 8 nm, in particular 5 nm.

11. A radiofrequency hyperthermal sensitiser agent comprising a nanoparticle comprising a non-conductive and non-magnetic matrix and metal cations with an atomic number Z greater than 40.

12. A method of treating a tumour by a radiofrequency radiation in a patient inducing hyperthermia of said tumour, comprising administering an effective does of nanoparticles into the tumour of said patient and exposing the tumour to radiofrequency radiation, wherein said nanoparticle comprises a non-conductive and non-magnetic matrix and metal cations having an atomic number Z greater than 40.
